(19) United States
(12) Patent Application Publication
(10) Pub. No.: US 2009/0104282 A1
(43) Pub. Date: Apr. 23, 2009

(54) METHOD FOR THE TREATMENT OF NAIL DISEASE

(76) Inventor: Natasha Aksenova, Brooklyn, NY (US)

Correspondence Address: URSULA B. DAY, ESQ., 708 Third Avenue, SUITE 1501, NEW YORK, NY, 10017, US

(21) Appl. No.: 12/340,251

(22) Filed: Dec. 19, 2008

Related U.S. Application Data
(63) Continuation of application No. 11/734,463, filed on Apr. 12, 2007, now abandoned.

(51) Int. Cl. A61K 33/40

(52) U.S. Cl. 424/616

(57) ABSTRACT

A treatment for diseased nails comprises treating the diseased nail after thorough cleaning with a peroxide solution, and thereafter applying an iodine solution until the diseased nail has recovered.

### BACKGROUND OF THE INVENTION

The present invention relates to a topical treatment and more specifically to a treatment for nail fungal and mold diseases.

Fungal and mold diseases are a scourge affecting a large number of the population. The disease can be contracted in a variety of ways especially in places where barefoot walking on wet surfaces is common such as gyms, pools, showers and similar. Even in places such as nail salons fungal disease can be contracted if the utensils are not autoclaved or sterilized by other means after every use.

The disease can originate from fungus, molds or dermatophytes representing about 50% of all nail disorders. There are a number of dermatophytes that cause most frequently toe nail infections. Among those, trichophyton rubrum and trychophyton mentagrophytes are the most familiar. But onychomycosis, ringworm of nails and tinea ungium can cause roughening, thickening and splitting of nails.

Oftentimes the fungal disease if caused by such biological agents such as epydermophyton, microsporum, trychophyton or ringworm producing nails that are opaque, white thickened and brittle.

Once the nail bed and the nail plate are infected, the victim's nails suffer from discoloration, onycholysis, and accumulation of subungual debris and nail plate dystrophy. The infections adversely affects the victims ranging from pain produced by footwear, general unsightliness of the nails; also loss of the nail due to the nail being dislodged form the nail bed due to inflammation and subsequent chafing off from pressure of footwear.

Treatment is most often by means of topical or oral use of antibiotics or antifungal agents. Examples of such agents are nyastin and amphotericin, or miconazole, clorimazole and others.

The treatment option is varied but in many cases the disadvantages of the treatment are very evident. The pre-treatment condition may return, or may not be abated since, for example, systemic antimicotic treatment agents require prolonged use and have potential for side effects. Other treatments may be impractical to use requiring soaking of the nail and such. Still other treatments are ineffective against the disease agent.

It would therefore be desirable and advantageous to provide an improved treatment to obviate prior art shortcomings and to provide a method for treatment which is simple in its application, which is readily available, easy to apply and providing an effective treatment for nails that are affected by any of the above described agents.

### SUMMARY OF THE INVENTION

According to one aspect of the present invention, the method for treatment of diseased nails comprises a method for treating nail plate and nail bed disease comprising: applying 3-5 drops of a first compound to the cleaned surface of the nail plate or nail bed; allowing this first solution to dry on the tissue and immediately thereafter applying 3-5 drops of a second compound to the nail plate or nail bed allowing it to dry; wherein the first compound is a solution of 3% of hydrogen peroxide and wherein the second compound is a Iodine tincture in 45% alcohol, and repeating these steps daily until the nail bed and the nail plate have recovered.

The present invention resolves prior art problems by allowing immediate treatment with agents that are easy to obtain and that are free from producing side effects.

Other features and advantages of the present invention will be more readily apparent upon reading the following description of currently exemplified embodiments of the invention as shown by the case studies.

The following case studies exemplify the application of the treatment and show its effectiveness.

### EXAMPLES

### Case I

A 40 year-old female had suffered from nail disease of the big toe on her right foot since the age of 25. The first symptoms started with cloudiness of the nail. Eventually, the nails changed color to bluish-black and yellow. The nail became thicker and thicker. While the upper layer of the nail was still shiny, the layers underneath were disintegrating. By her early thirties, the nail detached from the nail bed and the space under the toe nail became about 3-4 mm and wearing of tight shoes became painful. The subject was treated both abroad and in the United States with prescribed medication and external applications. The treating physician in the United States diagnosed her with candida and bacterial imbalance of the stomach and intestine and the subject took the prescribed medicine for over 2 years. None of the treatments seem to work. Then her primary care physicians insisted she terminate the medication since the medication could result in liver damage. The toe nail eventually completely detached during a pedicure session; however the newly growing toe nail showed the same symptoms as the previous one. The subject started treatment with the method according to the invention. By applying the two compounds daily. Each morning after her shower, she applied 4 drops of the first compound around the nail bed and under the nail. After a brief waiting time to allow the first compound to dry, the second compound was applied in the same areas as the first. About a month later, subject noted visible sign of change in the color of the nail. Within the course of about 3 months of the daily treatment, the toenail became pink, thin and completely attached. Within a year there has been no recurrence of the symptoms.

### Case II

A 30 year old male had a toenail on the left foot that was cracked vertically, which he noticed during clipping his nails. He did not recall that there was any source of trauma or injury. He allowed this condition for about 5 months without undertaking any treatment, when the toe nail became cloudy and painful. After seeing a physician, he took a prescription medicine which the subject took for at least 6 months. His physician had not carried out any tests. By perusing the internet, the subject learned of the frequency of toe nail disease and that the medication would have possible side effects and that there was no guarantee of a cure. The subject terminated the medication but applied some topical treatment which his physician had also recommended. While the topical treatment was not easy to carry out several times ad day in an office setting, he was offered treatment with compound No. 1 and No. 2, which he accepted. The application was the same as in case 1. 4-5 drops of the first compound in, around and under the toe nail followed by the same amount of the second compound. After about 4 months of treatment the nail had changed color and had become more transparent. Tight shoes no longer caused any more discomfort. While there was still a trace from the crack visible, the nail looked and felt healthy. No additional therapy has since been needed.

### CASE III

74 year female subject has suffered from persistent toe nail disease for the past 35 years. She did not remember what caused it. Her sister and her mother had the same problem as long as she could remember. The subject could hardly wear any shoes and had to use a locksmith file to decrease the thickness of the toenail. The toenail affected was the right big toe (same as the mother and sister) and the infection did not spread to the other toes. Various kinds of treatments were applied. Because nothing worked she had surgery to remove the toenail. However, the new toenail grew in exactly as the old one—diseased. When she started applying the compounds according to the method of the invention, she saw improvements within months. In approximately 2 months, the toenail had improved and the infection cleared up. No recurrence was recorded.

### Case IV

This subject was 70 years old and the sister of subject in case 3. The subject currently resides abroad. After subject of case 3 saw the positive results, she advised her sister to apply the same method. However, the daughter reported there was no improvement because the mother did not use the treatment regularly. The daughter then supervised the regular application of compound 1 and 2 and recently, the daughter reported that the mother is very happy since she could wear open shoes for the first time in years due to the greatly improved toe nail.

### Case V

A 65-year old pharmacist developed nail disease when working in Russia. It started on the nails of the right hand and spread to both hands. Even wearing gloves did not improve the condition. She started using a topical manganese compound to try and cure the problem; however, no improvement could be recorded. Her nails were always red and inflamed, they lost the glossy appearance and the nail plates were starting to break. The condition was such she had to wear gloves even while cooking at home. She started using the method of the present invention by applying 4-5 drop doses of the two compounds on a daily basis. It took several months for the nails to recover. After continued use, her nails have become shiny and transparent again.

### Case VI

The subject was a 59 year-old male who had hit his toenail during a weight lifting work-out. After some time, the toenail became discolored and then started to detach from the nail bed about 2 mm. He checked it daily with a toothpick. He started using the treatment daily after showering. After about 6 weeks he recorded a visible improvement.

### CASE VII

A young woman in her twenties had an infected toenail on her left foot causing considerable pain. Eventually the nail fell off. When it grew back the pedicurist noticed that the nail grew back in two layers. Applying pedicures to improve the situation did not help. The nail was dark and painful and the young women had to wear a prosthetic nail cover for some time. Several weeks after staring treatment with the No. 1 and No. 2 compounds, an improvement could be seen. The toe nail was cleared up after several months.

While the invention has been illustrated and described as embodied in the treatment of nails, it is not intended to be limited to the details shown since various modifications and structural changes may be made without departing in any way from the spirit of the present invention. The embodiments were chosen and described in order to best explain the principles of the invention and practical application to thereby enable a person skilled in the art to best utilize the invention and various embodiments with various modifications as are suited to the particular use contemplated.

What is claimed as new and desired to be protected by Letters Patent is set forth in the appended claims and their equivalents:

What is claimed is:

1. A method for treating nail plate and nail bed disease comprising:
applying 3-5 drops of a first compound to the cleaned surface of the nail plate or nail bed; allowing the first solution to dry on the tissue and immediately thereafter applying 3-5 drops of a second compound to the nail plate or nail bed allowing it to dry; wherein the first compound is a solution of 3% of hydrogen peroxide and wherein the second compound is an iodide tincture in 45% alcohol, and
repeating these steps daily until the nail bed and the nail plate have recovered.

2. The method of claim 1, wherein the nail is a toe nail.

3. The method of claim 1, wherein the nail is a finger nail.
